(19) United States
(12) Patent Application Publication
(10) Pub. No.: US 2009/0118531 A1
(43) Pub. Date: May 7, 2009

(54) NOVEL SUPPORTED CATALYST FOR AMMOXIDATION

(75) Inventors: Hartmut Hibst, Schriesheim (DE); Sabine Huber, Limburgerhof (DE); Frank Rosowski, Mannheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 12/298,806

(22) PCT Filed: Apr. 23, 2007
(86) PCT No.: PCT/EP2007/053938

§ 371 Date: Oct. 28, 2008

(30) Foreign Application Priority Data
Apr. 28, 2006 (EP) 06113313.8

(51) Int. Cl. C07C 253/28; B01J 23/22

(52) U.S. Cl. 558/329; 502/312

(57) ABSTRACT

Supported catalysts comprising a support having a mean diameter of ≦78 μm, a vanadium oxide, an antimony oxide, one or more alkali metal or alkaline earth metal oxides, and one or more oxides of tungsten, molybdenum, titanium, iron, cobalt, nickel, manganese, potassium, copper or mixtures thereof; processes for preparing said catalysts; and processes for preparing an aromatic or heteroaromatic nitrile in the presence of such a supported catalyst.

The present invention relates to a process for preparing an aromatic or heteroaromatic nitrile in the presence of a supported catalyst which comprises a support having a mean diameter of ≦78 μm. The present invention further relates to the new supported catalyst as such and to a process for preparing this new supported catalyst.

Processes for preparing aromatic nitriles, for example ortho-phthalonitrile (OPN) or isophthalonitrile (IPN) are known. Those processes which are carried out in the presence of a catalyst by reacting with ammonia and oxygen are also referred to as ammoxidation. For example, DE-A 21 64 401, DE-A 26 53 380 or EP-A 1 319 653 describe processes for preparing IPN starting from meta-xylene using chromium catalysts, IPN serving partly as an intermediate to prepare the corresponding diamino compound by hydrogenation. However, the use of chromium catalysts is problematic, since they are carcinogenic (chromate dusts) and thus also constitute a great environmental problem.

As alternatives to the chromium catalysts, aromatic nitrites are prepared especially by using catalysts which contain vanadium and/or antimony and may also, if appropriate, comprise further metals. For instance, EP-A 0 750 942 relates to particulate catalysts for use in a fluidized bed. The catalyst particles (the support) have a diameter in the range from 5 to 500 μm to an extent of 90 or more % by weight (percent by weight). Of these catalyst particles, those which have a diameter of from 20 to 75 μm in turn have a specific fracture resistance, expressed as fracture stress, which is expressed in a special formula. However, EP-A 0 750 942 does not disclose that the support material consists of particles which have a mean diameter of ≦78 μm.

EP-A 222 249 discloses a further process for preparing aromatic nitrites from alkyl-substituted aromatic hydrocarbons by catalytic oxidation with ammonia and oxygen or oxygen-comprising gases at elevated temperature in the vapor phase in the presence of a catalyst which comprises from 2 to 10% by weight of vanadium pentoxide, from 1 to 10% by weight of antimony trioxide, from 0.02 to 2% by weight of alkali metal oxide and from 0.01 to 1% by weight of alkaline earth metal oxide on alumina. The catalysts used therein have particle sizes having a diameter of from 0.05 to 0.3 mm; specific ranges with regard to the mean diameter of the particles are not disclosed in EP-A 222 249. This applies equally to the catalysts described in DE-A 37 00 710 which are used in a fluidized bed process.

EP-A 0 699 476 describes supported catalysts which are suitable for ammoxidation. The supported catalysts have, as the support material, essentially alumina, silica, titania and/or zirconia and, as the active composition, vanadium and antimony in oxidic form as essential components. The support material is spherical or approximately spherical and has a bulk density of from 0.6 to 1.2 kg/l. As further metal components of the active composition, the supported catalysts may, for example, comprise cesium and/or rubidium and tungsten, each in oxidic form. As a specific example, EP-A 0 699 476 describes the ammoxidation of ortho-xylene to OPN, the spherical alumina support used having a mean diameter of 150 μm.

EP-A 0 767 165 relates to a process for preparing aromatic or heteroaromatic nitrites using a very similar composition with regard to the active catalyst constituents (active composition) to the catalysts described in EP-A 0 699 476. The support of these supported catalysts described in EP-A 0 767 165 consists, however, of from 2 to 30 particle fractions whose mean diameter differs by from 10 to 80%, and the support has a bulk density of from 0.6 to 1.2 kg/l. As a specific example, the ammoxidation of ortho-xylene to OPN is described, and supports composed of mixtures of spherical alumina with mean diameters of 150 μm and 80 μm are used. However, EP-A 0 767 156 does not state that the mean diameter of the catalyst support particles may be ≦78 μm.

WO 05/28417 and WO 05/26104 describe further catalysts which can be used in the ammoxidation of meta-xylene to IPN. The catalysts described therein comprise vanadium, antimony and/or chromium, but no disclosures are made with regard to the mean diameter of the catalyst particles. In a specific working example, the ammoxidation of meta-xylene over a catalyst comprising vanadium, antimony, tungsten and cesium on steatite to IPN is described.

The object underlying the invention thus consists in the provision of an improved catalyst and of an improved process for preparing aromatic or heteroaromatic nitrites by ammoxidation.

According to the invention, this object is achieved by a new catalyst and a new and improved process using the new catalyst to prepare an aromatic or heteroaromatic nitrile of the general formula (I)

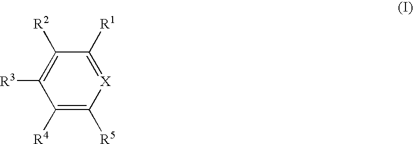

in which

X is nitrogen or C—R⁶ and

R¹,R²,R³,R⁴,R⁵

and R⁶ are each independently hydrogen, C₁-C₈-alkyl, halogen, trifluoromethyl, nitro, amino, cyano, C₁-C₇-cyanoalkyl, C₁-C₈-aminoalkyl or hydroxyl, with the proviso that at least one of the substituents is cyano or C₁-C₇-cyanoalkyl,

by reacting an aromatic or heteroaromatic hydrocarbon of the general formula (II)

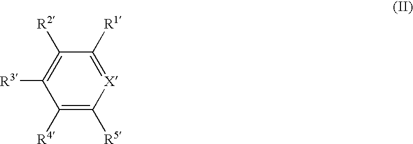

in which

X′ is nitrogen or C—R⁶′ and

R¹′,R²′,R³′, R⁴′,R⁵′

and R⁶′ are each independently hydrogen, C₁-C₈-alkyl, halogen, trifluoromethyl, nitro, amino, C₁-C₈-aminoalkyl or hydroxyl, with the proviso that at least one of the substituents is C₁-C₈-alkyl,

with ammonia and oxygen and/or an oxygen-comprising gas at a temperature of from 200 to 600° C. and a pressure of from 0.1 to 5 bar in the gas phase over a supported catalyst which comprises from 0.5 to 20% by weight of vanadium oxide, wherein the supported catalyst comprises a support having a mean diameter of ≦78 μm and the support has a bulk density of from 0.6 to 1.2 kg/l.

The process according to the invention and the inventive catalysts used therein have the advantage that, compared to the known prior art ammoxidation processes, a higher space-time yield can be achieved owing to the improved support geometry. This is achieved in an advantageous manner in the ammoxidation of aromatics, especially of ortho- and meta-xylene to OPN and IPN respectively. Even at a higher loading (higher xylene concentration in the feed and/or higher xylene throughput), the new catalysts can achieve higher xylene conversions and higher product of value selectivities in comparison to conventional catalysts. This has a positive effect on the costs arising for the dinitrile preparation. These advantages are exhibited especially when the inventive catalysts are used in a fluidized bed process, since the inventive catalysts exhibit improved fluidizing behavior owing to the smaller support geometry.

The process according to the invention can be carried out as follows:

A mixture of an aromatic or heteroaromatic hydrocarbon, ammonia and oxygen and/or an oxygen-comprising gas can be converted in the gas phase at temperatures of from 200 to 600° C., preferably from 300 to 550° C., more preferably from 350 to 500° C., and a pressure of from 0.1 to 5 bar, preferably from 0.3 to 2 bar, more preferably from 0.5 to 1.5 bar, especially at standard pressure (atmospheric pressure), in the presence of the inventive supported catalyst which is defined below. In the process according to the invention, the reactants are preferably reacted in a fluidized bed.

The starting compounds (aromatic or heteroaromatic hydrocarbons of the general formula (II)) are preferably taken up in a gas stream composed of ammonia, oxygen and/or an oxygen-comprising gas, their concentration being adjusted appropriately to from 0.1 to 25% by volume, preferably to from 0.1 to 10% by volume.

The present invention further provides supported catalysts as such which can be used in the process according to the invention. The inventive supported catalysts comprise

i) at least one support having a mean diameter of ≦78 μm and a bulk density of from 0.6 to 1.2 kg/l, preferably from 0.6 to 1.1 kg/l, more preferably from 0.7 to 1.0 kg/l, and

ii) from 0.5 to 20% by weight of vanadium oxide (calculated as vanadium(V) oxide).

As further components, the inventive supported catalysts may comprise:

iii) from 0 to 20% by weight of antimony oxide (calculated as antimony(III) oxide),

iv) from 0 to 4% by weight of one or more alkali metal or alkaline earth metal oxides, preferably cesium oxide, rubidium oxide or mixtures thereof especially cesium oxide, and

v) from 0 to 10% by weight of one or more oxides from the group of tungsten, molybdenum, titanium, iron, cobalt, nickel, manganese, potassium or copper.

In the inventive supported catalyst, the support makes up from 60 to 99% by weight of the total catalyst mass.

In the inventive supported catalysts, it is possible in principle to use all supports known to those skilled in the art, preferably supports of alumina, silica, titania, zirconia, silicon carbide, magnesia or mixtures thereof, preferably alumina, silica, titania, zirconia or mixtures thereof, more preferably alumina, silica or mixtures thereof, most preferably alumina.

Components ii) to v) are present in the inventive supported catalyst in the amounts which follow. The preferred amounts specified below for components iii) to v) relate only to those embodiments in which these optional components are present.

ii) from 0.5 to 20% by weight, preferably from 1 to 12% by weight, more preferably from 1 to 10% by weight, especially preferably from 3 to 7% by weight of vanadium oxide,

iii) from 0 to 20% by weight, preferably from 1 to 12% by weight, more preferably from 2 to 10% by weight, in particular from 4 to 9% by weight of antimony oxide,

iv) from 0 to 4% by weight, preferably from 0.2 to 3% by weight, more preferably from 0.5 to 1% by weight of one or more alkali metal or alkaline earth metal oxides, preferably cesium oxide, rubidium oxide or mixtures thereof, especially cesium oxide,

v) from 0 to 10% by weight, preferably from 0.01 to 5% by weight, more preferably from 0.1 to 3% by weight of one or more oxides from the group of tungsten, molybdenum, titanium, iron, cobalt, nickel, manganese or copper, preferably tungsten oxide or molybdenum oxide, especially tungsten oxide (calculated as tungsten(VI) oxide).

Components ii) to v) are present in the inventive supported catalysts in such amounts that the support (component i)) makes up from 60 to 99% by weight, preferably from 70 to 96% by weight, more preferably from 80 to 93% by weight of the total catalyst mass.

The inventive supported catalysts preferably do not comprise any chromium or chromium oxide, i.e. the inventive catalysts are chromium-free.

In a further preferred embodiment, the inventive supported catalysts comprise:

ii) from 3 to 7% by weight of vanadium oxide,

iii) from 4 to 9% by weight of antimony oxide,

iv) from 0.5 to 1% by weight of cesium oxide and/or

v) from 0.1 to 3% by weight of tungsten oxide.

The inventive supported catalysts comprise at least one support (component i)) having a mean diameter of ≦78 μm. The mean diameter (particle diameter) is also referred to as the D₅₀ value and defines the mean particle diameter of the individual support particles. This means that 50% by volume of the support particles have a smaller value than the mean diameter. The D₅₀ value is determined experimentally by means of a laser particle size analyzer from Cilas, Madison, Wis., USA. In addition, the particle size spectrum of the supports used is characterized by means of a sieve analysis. The mean diameter of the support particles is preferably from 20 to 78 μm, more preferably from 20 to 75 μm, even more preferably from 40 to 75 μm, especially preferably from 50 to 70 μm and very especially preferably from 60 to 65 μm.

The present invention further provides a process for preparing the inventive supported catalysts.

The supported catalysts can be prepared by simultaneous or successive saturation or impregnation of the support with one or more solutions and/or suspensions, preferably with one or more aqueous solutions or one or more aqueous suspensions of one or more compounds which comprise the active catalyst constituents such as vanadium and, if appropriate, antimony, tungsten, molybdenum, titanium, iron, cobalt, nickel, manganese, copper, alkali metal or alkaline earth metal, and subsequent drying and calcination, preferably under oxidizing conditions, at temperatures of from 400 to 800° C., preferably from 450 to 750° C. The impregnation solution or suspension is preferably not used in a larger amount than can be taken up by the support material. The impregnation can also be undertaken in several steps after intermediate drying in each case.

The saturation or impregnation solutions used are generally the active components, preferably in the form of aqueous solutions of their salts, especially of salts of organic acids which can be decomposed without residue in the oxidative calcination. Preference is given here to the oxalates, particularly in the case of vanadium, and to the tartrates, particularly in the case of antimony and tungsten, and the tartrates may also be present in the form of mixed salts, for example together with ammonium ions. To prepare such solutions, the metal oxides but also other metal compounds can be dissolved in the acids.

The substituents R¹, R², R³, R⁴, R⁵, R⁶, R¹′, R²′, R³′, R⁴′, R⁵′, R⁶′ and the intermediate member X and X′ in the general formula (I) or (II) are defined as follows:

X is nitrogen or C—R⁶, preferably C—R⁶,

X′ is nitrogen or C—R⁶′, preferably C—R⁶′,

R¹,R²,R³,R⁴,R⁵,R⁶,R¹′,R²′,R³′,R⁴′,R⁵′ and R⁶′ are each independently
hydrogen,
C₁-C₈-alkyl, preferably C₁-C₄-alkyl, more preferably methyl, ethyl, n-propyl and isopropyl,
halogen such as fluorine, chlorine, bromine or iodine, preferably fluorine, chlorine or bromine, more preferably chlorine or bromine, especially chlorine,
trifluoromethyl,
nitro,
amino,
C₁-C₈-aminoalkyl, preferably C₁-C₄-aminoalkyl, more preferably aminomethyl, 1-aminoethyl and 2-aminoethyl, and
hydroxyl,
with the proviso that at least one of the substituents in the general formula (II) is C₁-C₈-alkyl,

R¹,R²,R³,R⁴,R⁵,R⁶ are additionally each independently
cyano,
C₁-C₇-cyanoalkyl, preferably C₁-C₃-cyanoalkyl, more preferably cyanomethyl, 1-cyanoethyl and 2-cyanoethyl,
with the proviso that at least one, i.e. 1, 2, 3, 4, 5 or 6, preferably 1, 2 or 3, more preferably 1 or 2 of the substituents is cyano or C₁-C₇-cyanoalkyl.

In the above definitions, C₁-C₈-alkyl means, for example, that the corresponding alkyl radical has between 1 and 8 carbon atoms.

In one embodiment of the present invention, the compound of the formula (I) is preferably OPN or IPN, more preferably IPN.

Ammoxidation is of particular industrial significance for the preparation of OPN from o-xylene, of isophthalonitrile from m-xylene (meta-xylene), of terephthalonitrile from p-xylene (para-xylene), of benzonitrile from toluene and of nicotinonitrile from beta-picoline.

In the case of the xylenes, the ammoxidation of the first methyl group proceeds more rapidly than that of the second, so that it is also easily possible to obtain partial ammoxidation products, for example p-methylbenzonitrile from p-xylene, o-methyl-benzonitrile from o-xylene and, if appropriate, benzonitrile as a by-product.

The present invention will be illustrated with reference to the examples which follow.

### EXAMPLE 1

Preparation of a fluidized bed catalyst with the composition V₄Sb_{3.18}W_{0.38}Cs_{0.38}Oₓ In an externally heated 2 liter stirred apparatus, 237.1 g of oxalic acid dihydrate (from BASF AG, D-67056 Ludwigshafen; content of H₂C₂O₄.2 H₂O=99.75% by weight) are dissolved at 60° C. in 387.3 g of water with continuous stirring. 90.1 g of polyvanadate (from GfE, Gesellschaft für Elektrometallurgie, D-90431 Nuremberg; content of V₂O₅=89.5% by weight) are dissolved slowly in the solution, the temperature of the resulting mixture A rising to 90° C.

In a further externally heated 0.3 liter stirred apparatus, 16.8 g of cesium nitrate (from Chemetall, D-60323 Frankfurt; content of CsNO₃=98.2% by weight) are dissolved at 60° C. in 70 g of water with continuous stirring to obtain solution B.

In a further externally heated 2 liter stirred apparatus, 226.0 g of tartaric acid (from Brennkat GmbH, D-67663 Kaiserslautern; content of H₆C₄O₆=99.75% by weight) are dissolved at 60° C. in 210 g of water with continuous stirring. 102.7 g of antimony(III) oxide (from Antraco, D-10247 Berlin; Sb₂O₃ content=99.9% by weight) are added to the resulting solution which is heated to 90° C. Subsequently, 252.0 g of aqueous ammonia (from Bernd Kraft GmbH, D-47167 Duisburg; NH₃ content=25.0% by weight) are metered into the resulting suspension within 30 minutes, the resulting temperature rise being restricted to +2° C. by the rate of addition. The result is a virtually clear solution C.

Subsequently, the solution B at 60° C. is metered into the mixture A at 90° C. with continuous stirring. The resulting dark blue mixture D is heated to 90° C. within 15 minutes. 20.9 g of ammonium paratungstate hydrate (from H. C. Starck, D-3380 Goslar; content of WO₃=89.25% by weight) are added to the resulting mixture D which is stirred at 90° C. for 15 minutes. Subsequently, solution C is metered into the resulting mixture within 1 minute. The resulting mixture F is stirred at 90° C. for a further 10 minutes.

The support used is a Puralox support from Sasol, D-20537 Hamburg. The Puralox support consists of round alumina particles (Al₂O₃ content=96.5% by weight), has a specific BET surface area of 131 m²/g and has a bulk density of 0.78 g/cm³. The D₅₀ value of the particle diameter is 62.4 μm and exhibits, in the sieve analysis, the following particle size distribution:
particle diameter<25 μm=0.2% by weight
particle diameter<45 μm=16.9% by weight
particle diameter<90 μm=86.6% by weight

The pore volume of the support has a value of 0.38 cm³/g; the mean pore diameter is 11.7 nm. The water uptake capacity of the support is determined by the incipient wetness method and is 0.6 cm³/g.

2000 g of this Puralox support are introduced into a mixer (model RO2) from Eirich GmbH & Co KG, D-74732 Hardheim. With continuous mixing (rotating pot, mixing speed of the mixer-stirrer at level 1), the mixture E is metered into the mixer within 10 minutes, in the course of which the mixture E is absorbed fully by the initially charged Puralox support. Subsequently, the resulting powder is mixed further in the mixer at higher mixing speed of the mixer-stirrer (level 2).

The resulting dark gray powder is distributed on porcelain dishes with a bed height of 2 cm and dried in a drying cabinet at 100° C. overnight. The water uptake of the dried powder P is determined by the incipient wetness method to be 0.4 cm³/g.

In an externally heatable 1 liter stirred apparatus, 101.6 g of oxalic acid dihydrate (from BASF AG, D-67056 Ludwigshafen; content of H₂C₂O₄.2 H₂O=99.75% by weight) are dissolved at 60° C. in 166.0 g of water with continuous stirring, 38.6 g of polyvanadate (from GfE, Gesellschaft für Elektrometallurgie, D-90431 Nuremberg; content of V₂O₅=89.5% by weight) are dissolved slowly in the solution, the temperature of the resulting mixture A′ rising to 90° C.

In a further externally heatable 0.1 liter stirred apparatus, 7.2 g of cesium nitrate (from Chemetall, D-60323 Frankfurt; content of CsNO₃=98.2% by weight) are dissolved at 60° C. in 30 g of water with continuous stirring to obtain solution B′.

In a further externally heatable 0.5 liter stirred apparatus, 96.87 g of tartaric acid (from Brennkat GmbH, D-67663 Kaiserslautern; content of H₆C₄O₆=99.75% by weight) are dissolved at 60° C. in 90 g of water with continuous stirring. 44.0 g of antimony(III) oxide (from Antraco, D-10247 Berlin; Sb₂O₃ content=99.9% by weight) are added to the resulting solution which is heated to 90° C. Subsequently, 108.0 g of aqueous ammonia (from Bernd Kraft GmbH, D-47167 Duisburg; NH₃ content=25.0% by weight) are metered into the resulting suspension within 30 minutes, the resulting temperature rise being restricted to +2° C. by the rate of addition. The result is a virtually clear solution C′.

Subsequently, the solution B′ at 60° C. is metered into the mixture A′ at 90° C. with continuous stirring. The resulting dark blue mixture D′ is heated to 90° C. within 15 minutes. 8.94 g of ammonium paratungstate hydrate (from H. C. Starck, D-3380 Goslar; content of WO₃=89.25% by weight) are added to the resulting mixture D′ which is stirred at 90° C. for 15 minutes. Subsequently, solution C′ is metered into the resulting mixture within 1 minute. The resulting mixture E′ is diluted with 300 ml of water and stirred at 90° C. for a further 10 minutes.

A mixer (model RO2) from Eirich GmbH & Co KG, D-74732 Hardheim is charged with the powder P prepared above. With continuous mixing (rotating pot, mixing speed of the mixer-stirrer at level 1), the mixture E′ is metered into the mixer within 10 minutes, in the course of which the mixture is absorbed fully by the initially charged powder P. Subsequently, the resulting powder is mixed further in the mixer at higher mixing speed of the mixer-stirrer (level 2). The resulting dark gray powder is distributed on porcelain dishes with a bed height of 2 cm and dried in a drying cabinet at 100° C. overnight. Subsequently, the powder, in 200 g portions, is flowed over with 50 l (STP)/h of air in a rotating quartz glass sphere (rotation speed=8 rpm) in a rotary piston oven, heated to 330° C. within 1 hour, kept at 330° C. for 2 hours, heated to 560° C. within 1 hour and kept at 560° C. for 1 hour. Subsequently, the oven is switched off, so that the powder can cool in the rotating quartz glass sphere. The resulting yellow catalyst powder has a specific BET surface area of 127 m²/g and a pore volume of 0.33 cm³/g; the mean pore diameter is 16.4 nm. The bulk density is 0.81 g/cm³. The active composition present in the support material has the composition V₄Sb_{3.18}W_{0.38}Cs_{0.38}Oₓ. The weight fraction of the active composition in the catalyst (=support+active composition) is 13.3%. The specific density of the catalyst is 5.1 g/cm³.

### EXAMPLE 2

Comparative example for preparation of a fluidized bed catalyst:

The support used is Puralox from Sasol, consisting of round alumina particles having an Al₂O₃ content of 98.7% by weight. The support has a mean particle diameter D₅₀ of 150 μm. The sieve analysis leads to the following particle size distribution:
particle diameter<100 μm=2.5% by weight
particle diameter<200 μm=94.0% by weight
particle diameter<300 μm=99.2% by weight
particle diameter<500 μm=100% by weight.

The specific BET surface area is 129 m²/g. The pore volume has a value of 0.37 cm³/g; the mean pore diameter is 11.6 nm. The bulk density is 0.76 g/cm³.

The yellow catalyst powder prepared from this support analogously to example 1 and with the same chemical composition has a specific surface area of 121 m²/g. The pore volume is 0.33 cm³/g. The mean pore diameter is 16.6 nm. The bulk density has a value of 0.78 g/cm³.

### EXAMPLE 3

m-Xylene, air, ammonia and demineralized water are fed into an electrically heated fluidized bed reactor. If they are not already present in the gaseous state under standard conditions, all reactants are converted to the gaseous state beforehand by evaporation and introduced into the preheated fluidized bed reactor as an intimate mixture. The molar ratios of the reactants used are:

| Ratios of | mol/mol |
|---|---|
| NH₃:m-Xylene | 14 |
| NH₃:O₂ | 3.4 |
| O₂:m-Xylene | 4.1 |
| N₂:NH₃ | 1 |

In the fluidized bed reactor, 400 g of the catalyst from example 1 (D₅₀=62.4 μm) are installed. The m-xylene throughput is 280 g/h. The GHSV (gas hourly space velocity) is 4000/h. GHSV=[standard liters/(liters of catalyst·h)] with standard liters as the sum of all gaseous substances under standard conditions (25° C., 1 bar).

At a reactor temperature of 470° C., the following conversions (C)/selectivities (S) are obtained:
C (m-xylene)=99%
S (IPN)=81%
S (TN)=8%; TN=tolunitrile

### EXAMPLE 4

Comparative example for the use of a fluidized bed catalyst:

800 g of the catalyst from example 2 are installed in the fluidized bed reactor from example 3 (D₅₀=150 μm). The m-xylene throughput is 167 g/h. The GHSV is 1200/h.

At a reactor temperature of 470° C., the following conversions (C)/selectivities (S) are obtained:
C (m-xylene)=90%
S (IPN)=68%
S (TN)=14%

Example 4 shows that, with the same active composition on a support with D₅₀=150 μm, even with a distinctly lower GHSV value, significantly poorer catalytic properties are achieved.

1-13. (canceled)

14. A process comprising:
(a) providing an aromatic or heteroaromatic hydrocarbon of the general formula (II): 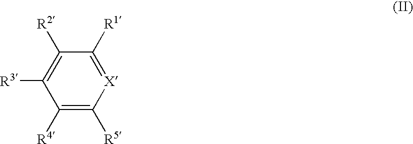
wherein X′ represents nitrogen or a C—R⁶′, and wherein R¹′,R²′,R³′, R⁵′ and R⁶′ each independently represents a substituent selected from the group consisting of hydrogen, C₁-C₈-alkyls, halogens, trifluoromethyl, nitro, amino, C₁-C₈-aminoalkyls, and hydroxyl, with the proviso that at least one of the substituents represents a C₁-C₈-alkyl;
(b) reacting the aromatic or heteroaromatic hydrocarbon of the general formula (II) with ammonia and oxygen or an oxygen-comprising gas at a temperature of 200 to 600° C. and a pressure of 0.1 to 5 bar in the gas phase over a supported catalyst;
wherein the supported catalyst comprises 0.5 to 20% by weight of a vanadium oxide, 1 to 12% by weight of an antimony oxide, 0.2 to 3% by weight of one or more alkali metal or alkaline earth metal oxides, and from 0.01 to 5% by weight of one or more oxides of a metal selected from the group consisting of tungsten, molybdenum, titanium, iron, cobalt, nickels, manganese, potassium, copper and mixtures thereof; and
wherein the supported catalyst comprises a support having a mean diameter of ≦78 μm and the support has a bulk density of 0.6 to 1.2 kg/l;
to provide an aromatic or heteroaromatic nitrile of the general formula (I): 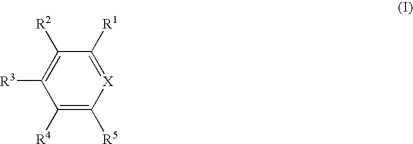
wherein X represents nitrogen or a C—R⁶, and wherein R¹,R²,R³,R⁴,R⁵ and R⁶ each independently represents a substituent selected from the group consisting of hydrogen, C₁-C₈-alkyls, halogens, trifluoromethyl, nitro, amino, cyano, C₁-C₇-cyanooalkyls, C₁-C₈-aminooalkyls, and hydroxyl, with the proviso that at least one of the substituents is cyano or a C₁-C₇-cyanoalkyl.

15. The process according to claim 14, wherein reacting the aromatic or heteroaromatic hydrocarbon of the general formula (II) with ammonia and oxygen or an oxygen-comprising gas is carried out in a fluidized bed.

16. The process according to claim 14, wherein the support comprises a material selected from the group consisting of alumina, silica, titania, zirconia and mixtures thereof.

17. The process according to claim 14, wherein the supported catalyst is chromium-free.

18. The process according to claim 14, wherein the supported catalyst comprises 3 to 7% by weight of the vanadium oxide, 4 to 9% by weight of the antimony oxide, 0.5 to 1% by weight of cesium oxide, rubidium oxide or mixtures thereof, and 0.1 to 3% by weight of tungsten oxide.

19. The process according to claim 14, wherein the aromatic or heteroaromatic nitrite of the general formula (I) comprises ortho-phthalonitrile (OPN), isophthalonitrile (IPN) or a mixture thereof.

20. A supported catalyst comprising:
(i) a support having a mean diameter of ≦78 μm and a bulk density of 0.6 to 1.2 kg/l;
(ii) 0.5 to 20% by weight of a vanadium oxide;
(iii) 1 to 12% by weight of an antimony oxide;
(iv) 0.2 to 3% by weight of one or more alkali metal or alkaline earth metal oxides; and
(v) 0.01 to 5% by weight of one or more oxides of a metal selected from the group consisting of tungsten, molybdenum, titanium, iron, cobalt, nickel, manganese, potassium, copper and mixtures thereof;
wherein the support is present in an amount of 60 to 99% by weight based on total catalyst mass.

21. The supported catalyst according to claim 20, wherein the support comprises a material selected from the group consisting of alumina, silica, titania, zirconia and mixtures thereof.

22. The supported catalyst according to claim 20, wherein the support has a mean diameter of 50 to 70 μm.

23. The supported catalyst according to claim 20, comprising
(ii) 3 to 7% by weight of the vanadium oxide;
(iii) 4 to 9% by weight of the antimony oxide;
(iv) 0.5 to 1% by weight of a cesium oxide; and
(v) 0.1 to 3% by weight of a tungsten oxide.

24. The supported catalyst according to claim 20, wherein the supported catalyst is chromium-free.

25. A process for preparing the supported catalyst according to claim 20, the process comprising: (a) saturating or impregnating the support simultaneously or successively with one or more solutions or suspensions comprising (ii) the vanadium oxide, (iii) the antimony oxide, (iv) the one or more alkali metal or alkaline earth metal oxides, and (v) the one or more oxides of a metal selected from the group consisting of tungsten, molybdenum, titanium, iron, cobalt, nickel, manganese, potassium, copper and mixtures thereof; and (b) drying and calcining the saturated/impregnated support at a temperature of 400 to 800° C.

26. The process according to claim 25, wherein calcining is carried out under oxidizing conditions.

27. The process according to claim 25, wherein the vanadium oxide comprises vanadium oxalate and the antimony oxide comprises antimony tartrate.
